(19) United States
(12) Patent Application Publication
(10) Pub. No.: US 2005/0021139 A1
(43) Pub. Date: Jan. 27, 2005

(54) Ophthalmic devices, methods of use and methods of fabrication

(76) Inventor: John H. Shadduck, Tiburon, CA (US)

Correspondence Address: John H. Shadduck, 1490 Vistazo West, Tiburon, CA, 94920, US

(21) Appl. No.: 10/890,576

(22) Filed: Oct. 7, 2004

Related U.S. Application Data
(63) Continuation-in-part of application No. 10/358,038, filed on Feb. 3, 2003.
(60) Provisional application No. 60/487,541, filed on Jul. 14, 2003.

(51) Int. Cl. A61F 2/16

(52) U.S. Cl. 623/6.35; 623/6.37; 623/6.56

(57) ABSTRACT

An adaptive optic for refractive lens exchange or cataract patients. The intracapsular implant comprises an elastomeric monolith with an equilibrium memory shape that imparts to the capsular sac's periphery the natural shape of the capsule in an accommodated state. In one embodiment, the monolith carries a recessed deformable central lens portion having an ultralow modulus that allows for high accommodative amplitude in response to equatorial tensioning. In a preferred embodiment, the adaptive optic defines an anisotropic modulus with a plurality of on-axis, rotationally symmetric elastomer block portions each having a different Young's modulus. The invention further provides composite materials for enhancing deformation of lens curvature, including the use of auxetic polymeric materials and negative stiffness materials. In preferred embodiments, at least a portion of the lens is fabricated of a shape memory polymer that provides a memory shape and a temporary shape with a reduced cross-sectional shape for introduction into the patient's eye.

### CROSS-REFERENCE TO RELATED APPLICATIONS

This application claims benefit of Provisional U.S. patent application Ser. No. 60/487,541 filed Jul. 14, 2003, titled Ophthalmic Devices, Methods of Use and Methods of Fabrication. This application also is a Continuation-in-Part of U.S. patent application Ser. No. 10/358,038 filed Feb. 3, 2003 titled Intraocular Implant Devices. The above applications are incorporated herein in their entirety by this reference.

### BACKGROUND OF THE INVENTION

1. Field of the Invention

The present invention is directed to intraocular adaptive optics and more specifically to an elastomeric optic that adapts (i.e., accommodates and disaccommodates) in response to normal physiologic zonular de-tensioning and tensioning forces. The adaptive optics are designed for cataract and refractive lens exchange procedures and combine with a post-phaco capsular sac to provide a biomimetic complex that mimics the energy-absorbing and energy-releasing characteristics of a still-accommodating lens capsule to alter the lens shape.

2. Description of the Related Art

The human lens capsule can be afflicted with several disorders that degrade its functioning in the vision system. The most common lens disorder is a cataract which consists of the opacification of the normally clear, natural crystalline lens matrix. The opacification usually results from the aging process but can also be caused by heredity or diabetes. FIG. 1A illustrates a lens capsule comprising a capsular sac with an opacified crystalline lens nucleus. In a typical cataract procedure as performed today, the patient's opaque crystalline lens is replaced with a clear lens implant or IOL. (See FIGS. 1A and 1B). The vast majority of cataract patients must wear prescription eyeglasses following surgery to see properly. Conventional IOLs in use today provide the eye with a fixed focal length, wherein focusing on both close-up objects and distant objects is not possible. Intraocular lens implantation for cataracts is the most commonly performed surgical procedure in elderly patients in the U.S. Nearly three million cataract surgeries are performed each year in the U.S., with an additional 2.5 million surgeries in Europe and Asia.

Mechanisms of Accommodation. Referring to FIG. 1A, the human eye has an anterior chamber 10 between the cornea 12 and iris 14 and a posterior chamber 20 between the iris and the lens capsule 101. The vitreous chamber 30 lies behind the lens capsule. The lens capsule 101 that contains the crystalline lens matrix LM or nucleus has an equator that is attached to cobweb-like zonular ligaments ZL that extend generally radially outward to the ciliary muscle attachments. The lens capsule 101 has transparent elastic anterior and posterior walls 104A and 104B that contain the crystalline lens matrix LM.

Accommodation occurs when the ciliary muscle CM contracts to thereby release the resting zonular tension on the equatorial region of the lens capsule 101. The release of zonular tension allows the inherent elasticity of the lens capsule to alter it to a more globular or spherical shape, with increased surface curvatures of both the anterior and posterior lenticular surfaces. The lens capsule together with the crystalline lens matrix and its internal pressure provides the lens with a resilient shape that is more spherical in an untensioned state. Ultrasound biomicroscopic (UBM) images also show that the apex of the ciliary muscle moves anteriorly and inward—at the same time that the equatorial edge the lens capsule moves inwardly from the sclera during accommodation.

When the ciliary muscle is relaxed, the muscle in combination with the elasticity of the choroid and posterior zonular fibers moves the ciliary muscle into the disaccommodated configuration, which is posterior and radially outward from the accommodated configuration. The radial outward movement of the ciliary muscles creates zonular tension on the lens capsule to stretch the equatorial region of lens toward the sclera. The disaccommodation mechanism flattens the lens and reduces the lens curvature (both anterior and posterior). Such natural accommodative capability thus involves contraction and relaxation of the ciliary muscles by the brain to alter the shape of the lens to the appropriate refractive parameters for focusing the light rays entering the eye on the retina-to provide both near vision and distant vision.

In conventional cataract surgery as depicted in FIGS. 1B and 1C, the crystalline lens matrix is removed leaving intact only the thin walls of the anterior and posterior capsules—together with zonular ligament connections to the ciliary body and ciliary muscles. The crystalline lens core is removed by phacoemulsification through a curvilinear capsularhexis as illustrated in FIG. 1B, i.e., the removal of an anterior portion of the capsular sac. FIG. 1B then depicts a conventional 3-piece IOL just after implantation in the capsular sac.

FIG. 1C next illustrates the capsular sac and the prior art 3-piece IOL after a healing period of a few days to weeks. It can be seen that the capsular sac effectively shrink-wraps around the IOL due to the capsularhexis, the collapse of the walls of the sac and subsequent fibrosis. As can be easily understood from FIGS. 1B and 1C, cataract surgery as practiced today causes the irretrievable loss of most of the eye's natural structures that provide accommodation. The crystalline lens matrix is completely lost-and the integrity of the capsular sac is reduced by the capsularhexis. The “shrink-wrap” of the capsular sac around the IOL can damage the zonule complex, and thereafter it is believed that the ciliary muscles will atrophy.

Prior Art Pseudo-Accommodative Lens Devices. At least one commercially available IOL, and others in clinical trials, are claimed to provide “accommodative” power adjustment even though the capsular sac shrink-wraps around the IOL as shown in FIG. 1C. Insofar as such prior art lens provides variable focusing power, it is best described as pseudo-accommodation since all the eye's natural accommodation mechanisms of changing the shape of the lens capsule are not functioning. Perhaps the most widely known of the pseudo-accommodative IOLs is a design patented by Cumming which is described in patent disclosures as having hinged haptics that are designed to flex even after the capsular sac is shrink-wrapped around the haptics. Cumming's patents (e.g., U.S. Pat. Nos. 5,496,366; 5,674,282; 6,197,059; 6,322,589; 6,342,073; 6,387,126) describe the hinged haptics as allowing the lens element to be translated forward and backward in response to ciliary muscle contraction and relaxation within the shrink-wrapped capsule. It is generally accepted that the movement of such lens is entirely pseudoaccommodative and depends on vitreous displacement that pushes the entire IOL slightly anteriorly. A similar IOL with four haptic arm elements is sold in Europe by HumanOptics, Spardorfer Strasse 150, 90154 Erlangen, Germany. The HumanOptics lens is the Akkommodative 1CU which is not available in the U.S., due to lack of FDA approval. In sum, any prior art IOLs that are implanted in an enucleated, shrink-wrapped lens capsule probably are not flexed by ciliary muscle relaxation, and exhibit only a pseudo-accommodative response due to vitreous displacement.

Since surgeons began using IOLs widely in the 1970's, IOL design and surgical techniques for IOL implantation have undergone a continuous evolution. While less invasive techniques for IOL implantation and new IOL materials technologies have evolved rapidly in the several years, there has been no real development of technologies for combining the capsular sac with biocompatible materials to provide a biomimetic capsular complex. What has stalled all innovations in designing a truly resilient (variable-focus) post-phaco lens capsule has been is the lack of sophisticated materials.

What has been needed are materials and intraocular devices that be introduced into an enucleated lens capsule through a 1 mm. to 2.5 mm. injector, wherein the deployed device and material provide the strain-absorbing and strain-releasing properties needed to transduce or amplify natural zonular tensioning and de-tensioning forces. Such an intraocular device will allow for the design of dynamic IOLs that can replicate natural accommodation. Microdevices of intelligent elastomeric polymers can provide the enabling technology to develop new classes of accommodating IOL systems.

### SUMMARY OF THE INVENTION

This invention relates to in-the-capsule implants having an anisotropic modulus for enhancing the accommodative amplitude of a lens component of the implant. The anisotropic properties are provided within nanoscale domains by molecular orientations or within microscale domains, for example, by soft lithography microfabrication methods. In preferred embodiments, the implants utilized a polymer monolith that includes shape memory polymers (SMPs) for allowing compact cross-sectional implant dimensions for introduction into the eye. The implants and accommodative lenses can be implanted using conventional techniques to create a biomimetic lens capsule complex. The capsular shaping components of the implants are designed to provide the implant/lens capsule complex with a shape, resiliency, and adaptive characteristics that mimic a young, still-accommodative lens capsule.

In one preferred embodiment, the intraocular lens (IOL) comprises an elastomeric monolith with a recessed central lens portion. The recessed lens allows for a steepened anterior curvature that can be subjected to both radial and axial deforming forces to amplify accommodative amplitude.

An exemplary IOL is configured for 360° intracapsular engagement of the lens capsule for preventing slippage between the implant and the lens capsule—to optimize force transduction to the elastomeric surfaces of the implant from zonular tensioning and de-tensioning. In most implant embodiments, a peripheral body portion of the implant is fabricated of a selected low modulus polymeric material that imparts resiliency and a memory shape to the lens capsule. The central adaptive optic portion of the implant is, at least in part, fabricated of an ultralow modulus polymeric material to provide greater amplitude of deformation or accommodation in response to forces transduced by the peripheral body portion from zonular excursion. An exemplary high amplitude adaptive optic can have a plurality of varied modulus portions in an on-axis, rotationally symmetric arrangement that can transduce limited equatorial forces into amplified deformation forces applied to the lens surfaces. In any embodiment of an adaptive optic corresponding to the invention, all or part of the lens can be fabricated of a shape memory polymer.

In another embodiment, the elastomeric intraocular lens monolith uses composite materials to provide novel and counterintuitive responses to stimuli in the form of zonular tensioning and de-tensioning. In one embodiment, a lens component comprises an auxetic material. An auxetic material has unique characteristics in that, when stretched lengthways, the material gets fatter rather than thinner (see FIGS. 2A and 2B). As well as this unique characteristic, auxetic materials have enhanced mechanical and physical properties, which means that they can actually be classified as both structural and functional materials.

Auxetic behavior is also known as a property that reflects a negative Poisson's ratio. Possion's ratio is defined as the ratio of the lateral contractile strain to the longitudinal tensile strain for a material undergoing uniaxial tension in the longitudinal direction. In other words, the Possion's ratio determines how the thickness of the material changes when it is stretched axially or lengthways. For example, when an elastic band is stretched axially the rubber material becomes thinner, giving it a positive Poisson's ratio. Elastomeric materials and solids typically have a Poisson's ratio of around 0.2-0.4. Poisson's ratio is determined by the internal structure of the materials.

In one example, FIG. 2A illustrates a two-dimensional cellular structure or honeycomb that is deformed uniaxially by hinge effects on ligaments 50 that form the cells 55 of the cellular network. For conventional hexagonal 2D cell geometry (see FIG. 2A), when the material is stretched along the x-axis, the cells 55 increase in dimension along the x- direction and decrease in dimension along the y-axis, giving a positive value for Poisson's ratio. Modifying the cellular geometry to adopt a “bow-tie” or “reentrant” structure as in FIG. 2B causes the material to have auxetic behavior, wherein upon uniaxial stretching the cellular network increases in dimension along both the x-axis and the y-axis.

Elasticity and hence auxetic behavior does not depend on scale. Elastic deformations can take place at domains ranging from the microscale to nanoscale (i.e., the molecular level). Within the molecular scale or domain, auxetic polymeric materials are known that have a node and fibril structure (see U.S. patent application Ser. No. 20030124279 by Sridharan et al, published Jul. 3, 2003, incorporated herein by reference). Thus, the scope of the invention encompasses these domains ranging from auxetic molecular materials to auxetic microfabricated structures.

The above described structures are elastically anisotropic—that is, they have a different Poisson's ratio depending on the direction in which they are stretched. The concepts underlying auxetic materials were first developed in isotropic auxetic foams by Roderic Lakes at the University of Wisconsin, Madison. Polymeric and metallic foams were made with Poisson's ratios as low as −0.7 and −0.8, respectively. Methods for scaling down honeycomb-like cellular structures include LIGA technology, laser stereolithography, molecular self-assembly, silicon surface micromachining techniques and nanomaterials fabrication processes. Auxetic two-dimensional cellular structures with cell dimensions of about 50 microns have been made by Ulrik Larsen et al. at the Technical University of Denmark. Three-dimensional microstructures consisting of two-dimensional conventional and auxetic honeycomb patterns on cylindrical substrates have been designed and fabricates by George Whitesides et al. at Harvard University (see Xu B., Arias F., Brittain S.T., Zhao X.-M., Grzybowski B., Torquato S., Whitesides G. M., “Making negative Poissons ratio microstructures by soft lithography”, Advanced Materials, 1999, v. 11, No. 14, pp. 1186-1189). Other background materials on auxetic materials are: Baughman, R, “Avoiding the shrink”, Nature, 425, 667, 16 Oct. (2003); Baughman, R, Dantas, S. Stafstrom, S., Zakhidov, A, Mitchell, T, Dubin, D., “Negative Poisson's ratios for extreme states of matter”, Science 288: 2018-2022, June (2000); Lakes, R. S., “Negative Poisson's ratio materials”, Science, 238 551 (1987); Lakes, R. S., “A broader view of membranes”, Nature, 414, 503-504, 29 Nov. (2001); Lakes, R. S., “Lateral Deformations in Extreme Matter”, perspective, Science, 288, 1976, June (2000); and Lakes, R. S., “No contractile obligations”, Nature, 358, 713-714, (1992). All these references are incorporated herein by reference.

In another embodiment, an implant corresponding to the invention is fabricated in part with inclusions of negative stiffness polymeric materials. Negative stiffness is characterized by a reversal of the usual co-directional relationship between force and displacement in deformable materials—such as an elastomeric monolith.

As background, a material's stiffness creates a force when something tries to deform it. This describes positive stiffness, that is, a material that pushes back when deforming forces are applied to alter the material's shape. A material with negative stiffness, in contrast, creates a force that amplifies the direction of deformation and the deformation force. Stated another way, force applied to deform an elastomeric body is in the same direction as displacement of the body, which equals positive stiffness. A reversal of these relationships corresponds to negative stiffness.

While negative stiffness is counter-intuitive, it does not violate any physical laws. Usually, however, negative stiffness in a material is unstable. In certain embodiments of the invention, negative stiffness or very low stiffness composites are disclosed in implants by means of inclusions of negative stiffness material in a polymeric monolith composite. The inclusions are adapted to store releasable energy in the body under certain deformations. Such inclusions of negative stiffness are stabilized in the composite by the surrounding matrix.

In another embodiment, it has been found that inclusions can comprise voids, cells or cavities in polymeric monoliths that can enhance deformations of surface curvatures of elastomeric monoliths. While voids, cells and cavities are not true negative stiffness materials, such inclusions in a monolith are considered herein as a functional components of a composite elastomeric material.

Negative stiffness differs from a negative Poisson's ratio as described above, in which lateral expansion occurs upon stretching.

Descriptions of negative stiffness materials are found in the following references, all of which are incorporated herein by this reference: Wang, Y. C. and Lakes, R. S., “Extreme stiffness systems due to negative stiffness elements”, American J. of Physics, 72, Jan. (2004); Wang, Y. C., Ludwigson, M., and Lakes, R. S., “Deformation of extreme viscoelastic metals and composites”, Materials Science and Engineering A, 370, 41-49, April (2004); Wang, Y. C. and Lakes, R. S., “Stable extremely-high-damping discrete viscoelastic systems due to negative stiffness elements”, Applied Physics Letters, 84, 4451-4453 (2004); Lakes, R. S. and Drugan, W. J., “Dramatically stiffer elastic composite materials due to a negative stiffness phase?”, J. Mechanics and Physics of Solids, 50, 979-1009 (2002); Lakes, R. S., “Extreme damping in compliant composites with a negative stiffness phase” Philosophical Magazine Letters, 81, 95-100 (2001); Lakes, R. S., “Extreme damping in composite materials with a negative stiffness phase”, Physical Review Letters 86, 2897-2900, 26 March (2001); Lakes, R. S., Lee, T., Bersie, A., and Wang, Y. C., “Extreme damping in composite materials with negative stiffness inclusions”, Nature, 410, 565-567, 29 March (2001); Rosakis, P., Ruina, A.; and Lakes, R. S., “Microbuckling instability in elastomeric cellular solids”, J. Materials Science, 28, 4667-4672 (1993).

Accordingly, invention advantageously provides an elastomeric monolithic IOL of an anisotropic structure that elastically engages the lens capsule in 360° for optimizing force transduction from zonular tensioning forces.

The invention advantageously provides polymer monolith with an ultralow modulus central optic portion that exhibits high amplitude dioptric changes in response to zonular tensioning.

The invention advantageously provides a polymer monolith that is at least in part fabricated of a shape memory polymer capable of self-deploying to a memory shape from a temporary reduced cross-sectional shape.

The invention provides a polymer monolith fabricated of a plurality of blocks of differing elastic moduli for enhancing accommodative amplitude in a recessed deformable lens surface.

The invention advantageously provides a polymer monolith with a peripheral body portion including at least one of auxetic materials, inclusions of open cells and inclusions of negative stiffness materials for enhancing accommodative amplitude.

These and other objects of the present invention will become readily apparent upon further review of the following drawings and specification.

### BRIEF DESCRIPTION OF THE DRAWINGS

In order to better understand the invention and to see how it may be carried out in practice, some preferred embodiments are next described, by way of non-limiting examples only, with reference to the accompanying drawings, in which like reference characters denote corresponding features consistently throughout similar embodiments in the attached drawings.

FIG. 1A is a perspective cut-away view of an eye with an opacified lens capsule.

FIG. 1B is a perspective cut-away view of the eye of FIG. 1A with a curvilinear capsularhexis and the crystalline lens matrix removed by phacoemulsification, together with the implantation of a prior art 3-piece IOL.

FIG. 1C is a perspective cut-away view of the eye of FIG. 1B showing the lens capsule after wound healing wherein the lens capsule shrink-wraps around the prior art IOL.

FIG. 2A provides schematic views of a material in unstretched and stretched configurations exhibiting a positive Poisson's ratio.

FIG. 2B provides schematic views of an auxetic material in unstretched and stretched configurations exhibiting a negative Poisson's ratio.

FIG. 3 provides schematic views of an auxetic molecular structure.

FIG. 4A is a perspective cut-away view of a lens capsule with an elastomeric monolithic implant corresponding to the invention implanted therein.

FIG. 4B is a sectional view of the elastomeric monolithic implant of FIG. 4A showing an equilibrium accommodative shape and a disequilibrium disaccommodative shape.

FIG. 5 is a sectional view of an alternative elastomeric monolith similar to that of FIGS. 4A-4B with a posterior surface portion.

FIG. 6A is the cross-sectional shape of a natural lens capsule in an equilibrium accommodative shape.

FIG. 6B is the cross-sectional shape of an exemplary elastomeric monolithic implant with a recessed central optic portion.

FIG. 7 is a sectional schematic view of an elastomeric monolith with block portions having different Young's moduli in an equilibrium shape.

FIG. 8A is an illustration of the haptics of a prior art intraocular lens that relies on a hinge mechanism for responding to zonular tensioning and de-tensioning forces, and that does exhibit elastic intracapsular engagement.

FIG. 8B is an illustration of another prior art haptics that relies on a hinge mechanism for responding to zonular tensioning and de-tensioning forces, and that does exhibit elastic intracapsular engagement.

FIG. 9 is a sectional perspective view of an alternative elastomeric monolith with a recessed lens portion and elastomer block portions having different Young's moduli in equilibrium and disequilibrium shapes.

FIG. 10 is a sectional view of an alternative elastomeric monolith similar to FIG. 9 with a plurality of elastomer block portions having different Young's moduli to provide a gradient modulus.

FIG. 11A is a sectional view of a portion of an elastomeric monolith similar to FIGS. 7, 9 and 10 with an auxetic material symmetrically carried in a peripheral region of the implant for enhancing accommodative amplitude, the monolith in an accommodative shape.

FIG. 11B is a sectional view as in FIG. 11A showing the elastomeric monolith in a disaccommodative shape after zonular tensioning wherein the auxetic material elevates and flattens the peripheral lens curvature.

FIG. 12A is a sectional view an alternative elastomeric lens monolith with inclusions of voids in a peripheral region of the optic portion, the monolith in an accommodative shape.

FIG. 12B is a sectional view showing the elastomeric monolith of FIG. 12A in a disaccommodative shape after zonular tensioning wherein the voids cause elevation and flattening of the peripheral lens curvature.

FIG. 13 is a sectional view of an alternative elastomeric monolith that has negative stiffness inclusions in a peripheral elastomer block for amplifying deformations caused by zonular tensioning forces, the lens shown on accommodative and accommodative shapes.

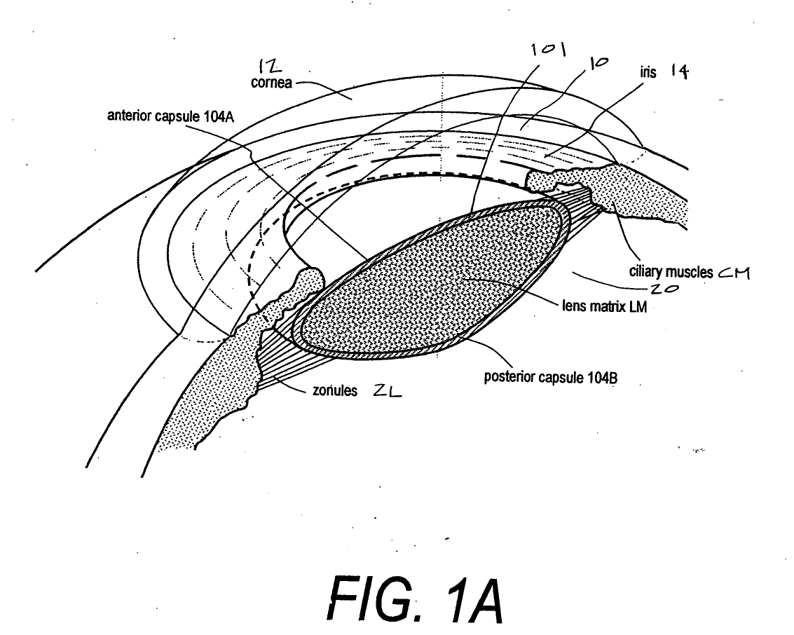

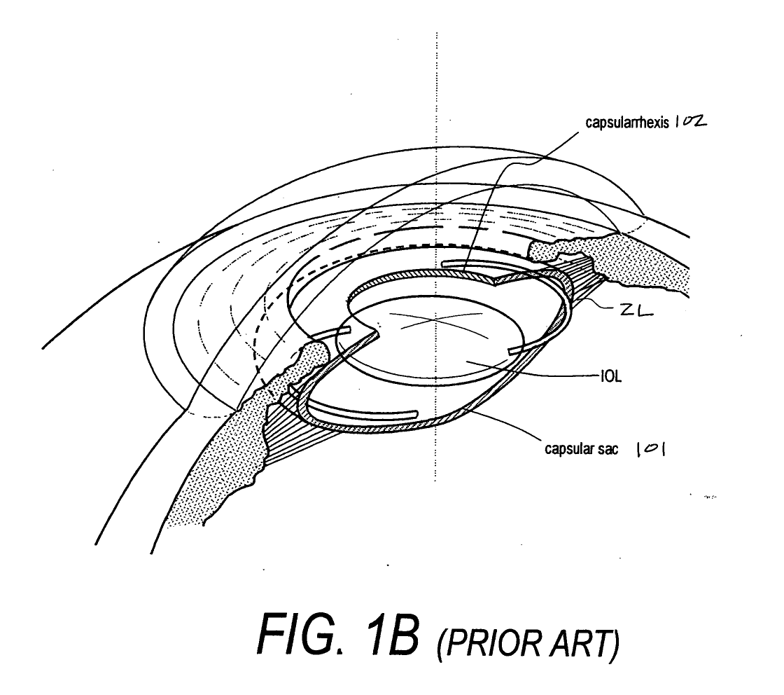

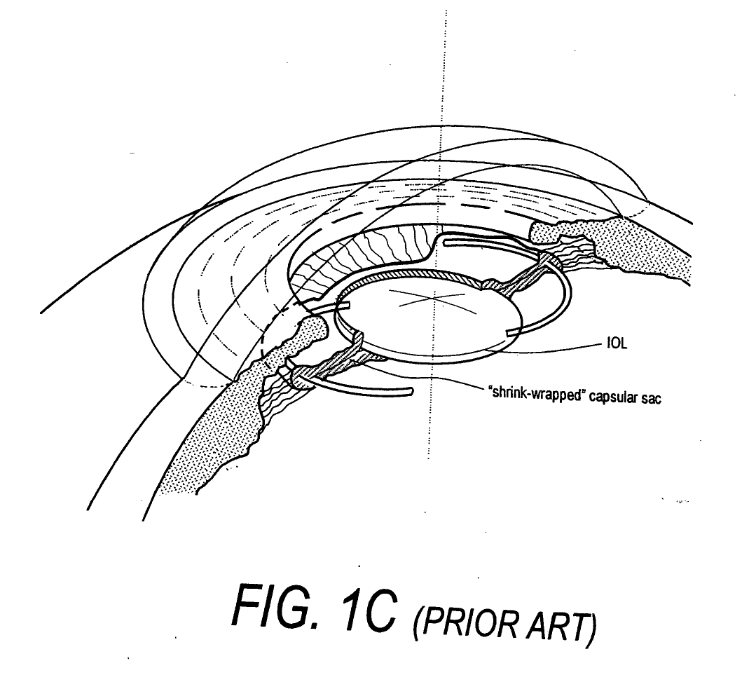

### DETAILED DESCRIPTION OF THE PREFERRED EMBODIMENTS

The adaptive optic implants corresponding to the invention are designed to create a biomimetic lens capsule complex that cooperates with zonular tensioning and de-tensioning forces. The term biomimetic lens capsule is derived from the word biomimesis, which defines the development of structures that mimic life, or that imitate biological systems. In preferred embodiments, the implant defines 360° elastic engagement with the capular sac and mimics the inherent elastic response of a still accommodative lens capsule. The implant thus cooperates with the ciliary muscles to alter the shape and power of a lens component of the implant.

The biomimetic lens complex is provided by combining the lens capsule with an elastomeric polymer monolith that engages at least the periphery of the capsule. The exemplary implant embodiments can comprise an isotropic polymeric material, and in preferred embodiments comprises a microfabricated polymer structure or molecular structure that provides anisotropic properties to the implant. In preferred embodiments, at least a portion of the monolith is fabricated of a solid shape memory polymer. Alternatively, a portion of implant also can be of a shape memory polymer foam.

Shape Memory Polymer Background. In preferred embodiments of intracapsular implants corresponding to the invention, an implant is made in part, or in its entirety, from a class of shape memory polymer (SMP). The term “shape memory” has a meaning in the context of SMPs that differs from the more common use of the term used in superelastic shape memory alloys, i.e., nickel titanium alloys. As background, a shape memory polymer is said to demonstrate shape memory phenomena when it has a fixed “temporary” shape that can revert or self-deploy to a “memory” shape upon a selected stimulus, such as temperature. A shape memory polymer generally is characterized as defining phases that result from glass transition temperatures in segregated linear block co-polymers: a hard segment and a soft segment. The hard segment of a SMP typically is crystalline with a defined melting point, and the soft segment is typically amorphous, with another defined transition temperature. In some embodiments, these transitions need not be related to glass transitions or melt temperatures.

In one embodiment, when the SMP material is elevated in temperature above a glass transition temperature or other transition temperature of the hard segment, the material then can be formed into a memory shape. The selected shape is memorized by cooling the SMP below the selected transition temperature of the hard segment. When the shaped SMP is cooled below the transition temperature of the soft segment while the shape is deformed, that (temporary) shape is fixed. The original shape is recovered by heating the material above a selected transition temperature of the soft segment but below the transition temperature of the hard segment. (Other methods for setting temporary and memory shapes are known which are described in the literature below). The recovery of the memory original shape is thus induced by an increase in temperature, and is termed the thermal shape memory effect of the polymer. The temperature can be at or below body temperature (37° C.) or a selected higher temperature.

Besides utilizing the thermal shape memory effect of the polymer, the memorized physical properties of the SMP can be controlled by its change in temperature or stress, particularly in ranges of the melting point or glass transition temperature of the soft segment of the polymer, e.g., the elastic modulus, hardness, flexibility, permeability and index of refraction. The scope of the invention of using SMPs in intracapsular implants extends to the control of such physical properties, particularly in the elastic composite structures described further below.

Examples of polymers that have been utilized in hard and soft segments of SMPs include polyurethanes, polynorborenes, styrene-butadiene co-polymers, cross-linked polyethylenes, cross-linked polycyclooctenes, polyethers, polyacrylates, polyamides, polysiloxanes, polyether amides, polyether esters, and urethane-butadiene co-polymers and others identified in the following patents and publications: See, e.g., U.S. Pat. No. 5,145,935 to Hayashi; U.S. Pat. No. 5,506,300 to Ward et al.; U.S. Pat. No. 5,665,822 to Bitler et al.; and U.S. Pat. No. 6,388,043 to Langer et al. (all of which are incorporated herein by reference); Mather, Strain Recovery in POSS Hybrid Thermoplastics, Polymer 2000, 41(1), 528; Mather et al., Shape Memory and Nanostructure in Poly(Norbonyl-POSS) Copolymers, Polym. Int. 49, 453-57 (2000); Lui et al., Thermomechanical Characterization of a Tailored Series of Shape Memory Polymers, J. App. Med. Plastics, Fall 2002; Gorden, Applications of Shape Memory Polyurethanes, Proceedings of the First International Conference on Shape Memory and Superelastic Technologies, SMST International Committee, pp. 115-19 (1994); Kim, et al., Polyurethanes having shape memory effect, Polymer 37(26):5781-93 (1996); Li et al., Crystallinity and morphology of segmented polyurethanes with different soft-segment length, J. Applied Polymer 62:631-38 (1996); Takahashi et al., Structure and properties of shape-memory polyurethane block copolymers, J. Applied Polymer Science 60:1061-69 (1996); Tobushi H., et al., Thermomechanical properties of shape memory polymers of polyurethane series and their applications, J. Physique IV (Colloque Cl) 6:377-84 (1996)) (all of the cited literature incorporated herein by this reference). Also, see Watt A. M., et al., Thermomechanical Properties of a Shape Memory Polymer Foam, available from Jet Propulsion Laboratories, 4800 Oak Grove Drive, Pasadena Calif. 91109 (incorporated herein by reference). SMP foams function in a similar manner as the shape memory polymers described above. The scope of the invention extends to the use of SMP foams for use in any elastic composite structures, for example the peripheral portion of the implant that does not need to be optically transparent.

Other derivatives of SMPs that fall within the scope of the invention fall into the class of bioerodible shape memory polymers that again may be used in elastic composite structures. For example, one embodiment of intracapsular implant can be designed with composite portions that define a first modulus of elasticity and shape for a period of time after implantation to better engage with capsular sac (e.g., under cyclopegia) followed by a transition to a second modulus of elasticity and memory shape following a selected time period to provide a lower modulus adaptive optic.

In all variants of intracapsular implants described herein, the principal objectives relate to the design of an implant that will impart to the implant/capsular sac complex an unstressed more spherical shape with a lesser equatorial diameter when zonular tension is relaxed, and a stressed flatter shape with a greater equatorial diameter in response to zonular tensioning forces. The resilient implant provides the ability to absorb known amounts of stress—and can release the energy in millions of cycles over the lifetime of the implant to thereby deform an elastomeric optic to provide variable focus.

Exemplary Biomimetic Implants and Adaptive Optics. In general, the biomimetic polymer implants corresponding to the invention define a first body portion or block that is adapted to engage the interior periphery of a capsular sac in 360°. Most often, this peripheral body comprises a non-optic portion of the lens system and imparts the desired memory shape to the biomimetic lens capsule. Most important, the peripheral implant body play roles as (i) a force transduction mechanism and as (ii) an actuation mechanism to “actuate” the central adaptive optic portion of the lens system. In preferred embodiments, the peripheral (first) and central (second) portions of the lens systems are body portions of a polymer monolith. In other embodiments, the first and second body portions of the lens system are independent components that mate after being independently introduced into the capsule.

In exemplary embodiments, the designs utilize a peripheral implant body that is, at least in part, fabricated of a selected higher modulus polymeric material that imparts resiliency and memory shape to the lens capsule. The central adaptive optic portion of the implant is, at least in part, fabricated of an ultralow modulus polymeric material that allows high amplitude of deformation or accommodation in response to forces transduced by the peripheral body portion from zonular excursion. An exemplary high amplitude adaptive optic can comprise a composite of varied modulus portions that define an on-axis, rotationally symmetric anisotropic properties (e.g., stiffness) that transduce limited equatorial forces into amplified deformation forces to alter curvatures of the lens surfaces. In any of the adaptive optic designs, all or part of the lens can be fabricated of a shape memory polymer, as described above, to provide for a compacted cross-section for introduction, or for post-implant adjustment of a parameter of a selected portion of the lens. For example, the SMPs can be used for post-implant adjustment of the modulus of a selected region, for modification of non-optic surface morphology and the like.

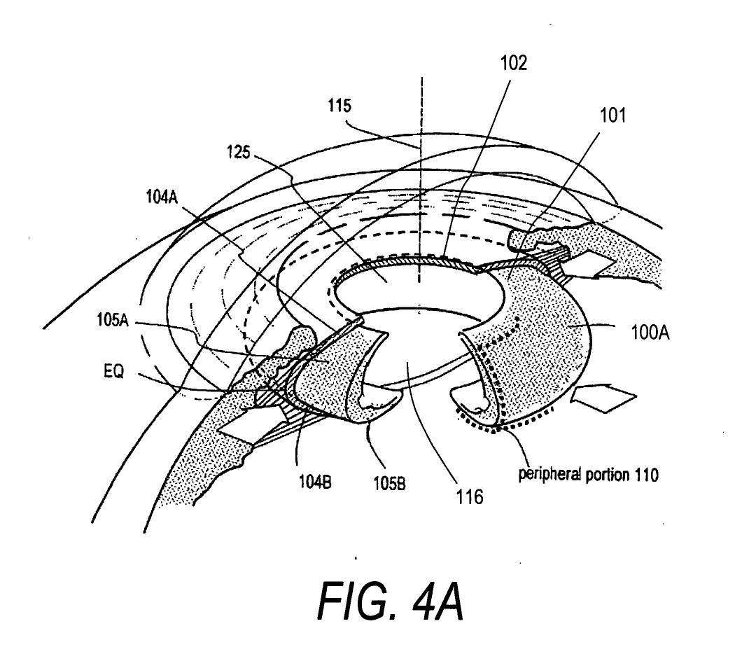

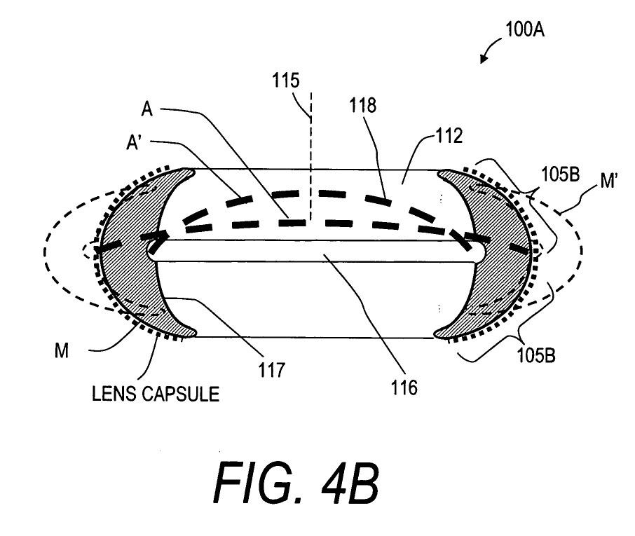

FIGS. 4A and 4B illustrate sectional views of a resilient polymer implant body 100A corresponding to the present invention, with FIG. 4A depicting the implant body 100A in a post-phaco lens capsule 101. In FIG. 4A, the intracapsular implant 100A is shown in an unstressed state, or memory shape, that is configured to maintain the lens capsule in an open spherical shape. In this disclosure, the term “lens capsule” refers to the thin membrane or structure that surrounds the interior lens matrix. In FIG. 4A, the capsule 101 has a capsularhexis 102 in the anterior capsule 104A for removal of the crystalline lens matrix. The posterior capsule is indicated at 104B. This disclosure will adopt the terminology in common use by ophthalmologists that defines the anterior capsule as the portion of the capsule that is anterior to the capsular equator region EQ, and defines the posterior capsule as the portion that is posterior to the equatorial region.

In FIG. 4A, the implant body 100A comprises an elastomeric monolith that generally can be defined as having anterior peripheral surface region 105A that engages the post-capsularhexis anterior capsule 104A and a posterior surface region 105B that engages at least a portion of the posterior capsule 104B. Referring again to FIG. 4A, it can be seen that implant body 100A has peripheral circumferential surfaces 105A and 105B collectively indicated at 110 that elastically engage the inner equatorial and peripheral surfaces of the capsular sac in 360°. As can be seen in FIG. 4A, the peripheral surfaces 110 of the three-dimensional implant body 100A thus impart “shape” to the lens capsule in its accommodative shape. It is the combination of implant body 110 and the capsule 101 that provides a biomimetic lens capsule that cooperates with the eye's natural accommodation mechanisms to enable new classes of accommodating lens systems. This implant/capsule complex can thus mimic a naturally accommodative human lens capsule in force transduction. Further, the elastomeric monolithic body 100A provides for peripheral shape changes intermediate its equilibrium memory shape and its temporary stressed, disequilibrium shape that correspond to natural lens capsule shapes. FIG. 4B illustrates implant body 100A in its memory shape with a phantom view of the implant body in a temporary stressed shape.

The implant of FIG. 4A comprises an annular elastomeric monolith that is dimensioned to engage the peripheral portion of the capsular sac around a central open portion 112 and optical axis 115. As can be seen in FIGS. 4A and 4B, the annular body 100A has an arcuate interior sectional shape 117 about open central portion 112 and axis 115, which is adapted to receive a drop-in IOL as will be described below. An interior annular engagement or receiving structure 116 is adapted to receive a peripheral haptic portion of a modular drop-in IOL 118 (phantom view in FIG. 4B). For example, two, three or four haptic arm elements or a 360° member can engage the structure 116 by springably engaging a groove, or by locking into a groove with any suitable lock-in structure.

As depicted in FIG. 4B, the monolithic body 100A has a selected modulus of elasticity for cooperating with natural accommodating forces cause by zonular tensioning, which can flatten the body 100A and alter its shape from M to M′. At the same time, the modular IOL 118 (phantom view) carried therein can be altered from shape or position A to A′, which can consist of either or both axial lens translation and lens curvature change.

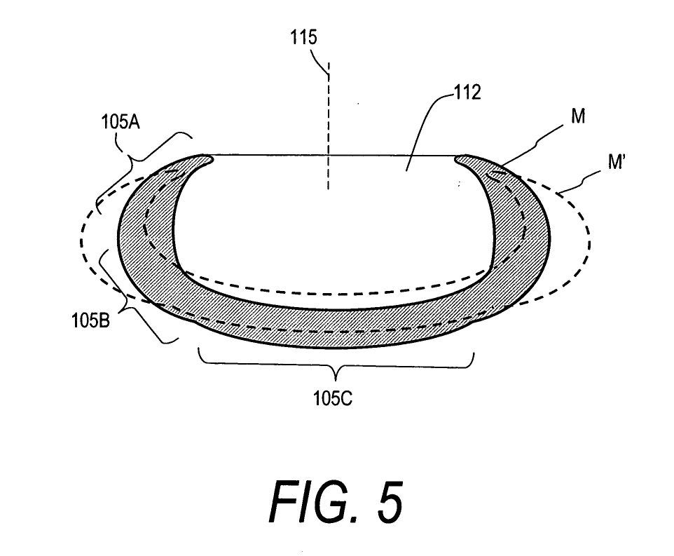

Preferably, the monolithic body 100A is fabricated of a shape memory polymer as described above. A preferred implant corresponding to the invention is fabricated of an open cell microfabricated shape memory polymer, a foam SMP or a substantially fluid impermeable solid SMP. The use of any SMP will allow for self-deployment of the implant to the memory implant shape as in FIG. 4A from a reduced cross section temporary shape. The polymer in its memory shape has an elastic modulus or Young's modulus of less than about 200 KPa. More preferably, the elastic modulus is less than about 100 KPa, and still more preferably less than about 50 KPa. Stated another way, the intracapsular device 100A is provided with a selected modulus to cooperate with the force of contraction of the human ciliary muscle and zonular tensioning which comprises a few grams of force about the equator of the capsular sac. The implant or adaptive optic deforms in response to less that about 2.5 grams of force. In other words, the contracting forces of the ciliary muscle will be sufficient to deform the intraocular device to provide the lens capsule complex with a lesser axial dimension—i.e., a flatter shape. Upon relaxation of the ciliary muscle and zonular tensioning about the equator of the capsular sac, the intraocular device defines recoverable strain properties (see arrows in FIG. 4A) that will return the implant to its non-stressed or memory shape wherein the implant has a greater axial dimension—i.e., a more spherical or globular shape. In another embodiment, as depicted in FIG. 5, the posterior surface region 105B is continuous in central region 105C and extends across the entire central region of a posterior capsule 104B (see FIG. 4A).

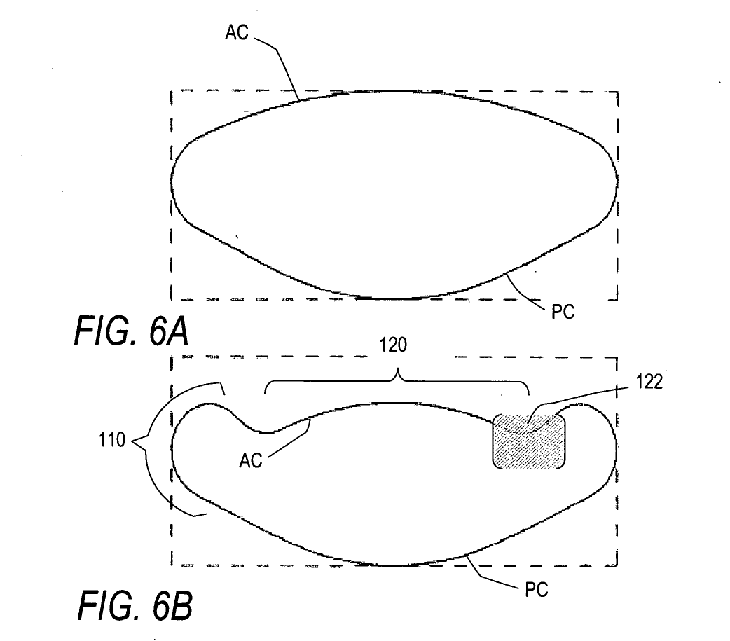

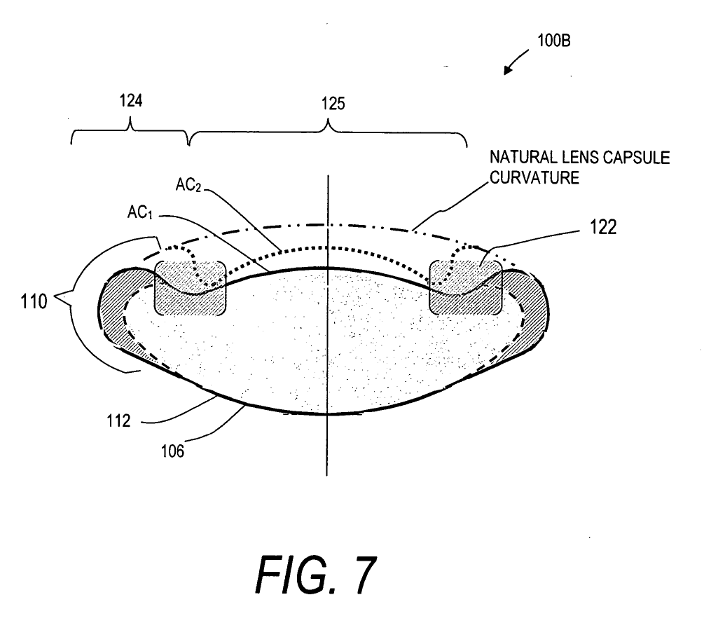

Now turning to FIGS. 6A and 6B, implant systems are described that comprise a unitary elastomeric monolithic implant 100B that includes a central lens or optic portion. FIG. 6A illustrates an exemplary profile of an intact young lens capsule having a natural accommodated lens curvature. FIG. 6B depicts the cross section of an exemplary elastomeric monolithic implant 100B, wherein the implant includes the functionality described above in implant 100A, and further includes a recessed central region 120 that defines a 360° peripheral notch region or arcuate interface 122 that transitions the annular peripheral body portion 124 into the deformable lens or optic portion indicated at 125 (FIG. 7). It can be seen in FIGS. 6A and 6B that the anterior curvature AC and posterior curvature PC of the recessed lens implant can correspond to the shape of a natural lens capsule in its accommodated state.

The elastomeric monolith 100B of FIGS. 6B and 7 has a non-optic portion 124 and a central optic region 125 about 4.5 mm. to 7 mm. in diameter. Of particular interest, the recessed region and the “notch” or arcuate interface 122 has been designed to provide multiple functionality. In one function, the anterior curvature AC of the optic monolith portion can be steepened when compared to the natural anterior curvature of FIG. 6A. If such a steep anterior curvature were provided in a continuous smooth curve (cf. FIG. 6A), the curvature would impinge upon the iris. Increased accommodative amplitude can be provided in the deformation of such a hypersteepened lens. FIG. 7 illustrates implant body 100B with a range of shapes (anterior curvatures AC₁, AC₂) that fall within the scope of the invention. In another function, the recessed region and in particular the arcuate interface 122 provide a design feature that can be controllably deformed to further amplify accommodative amplitude. The recessed region further advantageously reduces the volume of the implant to allow for easier folding or compaction for introduction into the eye through a small diameter introducer.

Preliminarily, the peripheral surfaces 110 of the monolith are again adapted for elastic engagement with the lens capsule-and correspond to the surfaces described in the implant embodiment of FIGS. 4A and 4B. As will be described below, the implant's elastomeric surface 110 and method of the invention (whether the implant cooperates with a drop-in lens or comprises an integrated optic monolith) for the first time (i) provides a omni-directionally deformable surface in which 100% of peripheral surface region 110 comprises a force-transduction structure to apply and respond to zonular forces on the capsular sac 101 to allow movement of the implant-capsule complex between its accommodated and disaccommodated shapes.

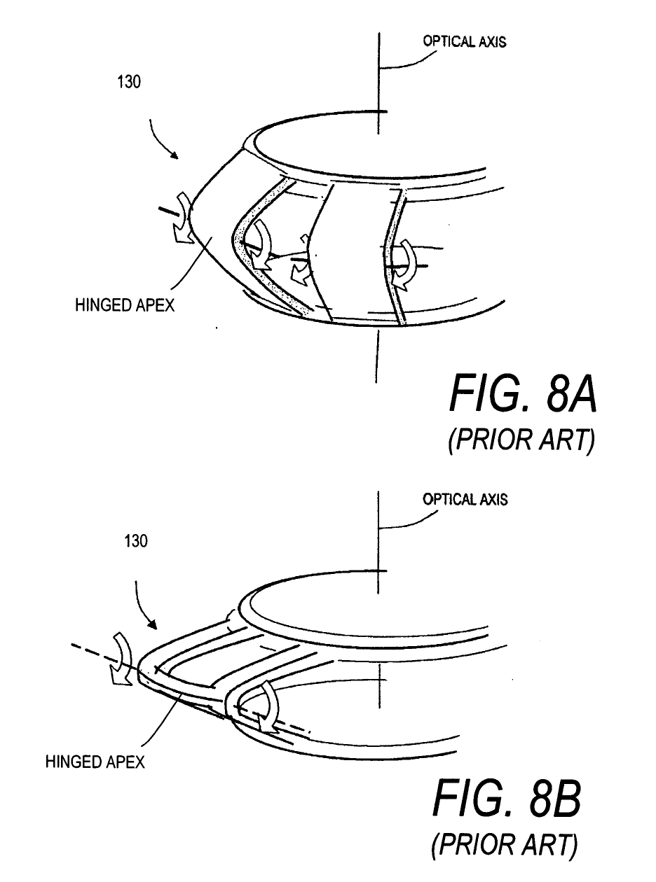

Of particular interest, this aspect of the present invention distinguishes the implant body 100B from other IOL implants in the patent literature that prop open a lens capsule. In the patent literature and published patent disclosures, the prior art designs use one form or another of a “leaf spring” member 130 that bends or flexes about a hinged apex within the capsular sac to apply and receive only 2D forces as indicated in FIGS. 8A and 8B. The IOL designs as in FIGS. 8A and 8B by their very nature “slip” within the lens capsule and thus cannot optimize force transduction from the zonules through the capsule to the implant and vice versa. These IOL designs do not provide an implant body configured for 360° elastic intracapsular engagement of the capsule periphery to provide effective means for force transduction. The illustration of FIG. 8A depicts the leaf-spring type haptics of the lens designs of Woods in U.S. Pat. Nos. 4,790,847; 6,217,612; 6,299,641 and 6,443,985 and Sarfarazi in U.S. patent application Ser. Nos. 20040015236; 20030130732; 20020045937 and 20020002404. The illustration of FIG. 8B depicts the leaf-spring type haptics of Zadno-Azizi et al in U.S. patent application Ser. Nos. 20030078657; 20020173847; 20020116061; 20020116058; 20020116057 and 20020107568. Finite element analyses indicate that the implant-capsule slippage factor plays a significant role in limiting force transduction, particularly at the scale of the implant and in relation to the limited forces that must be captured and transduced by the system.

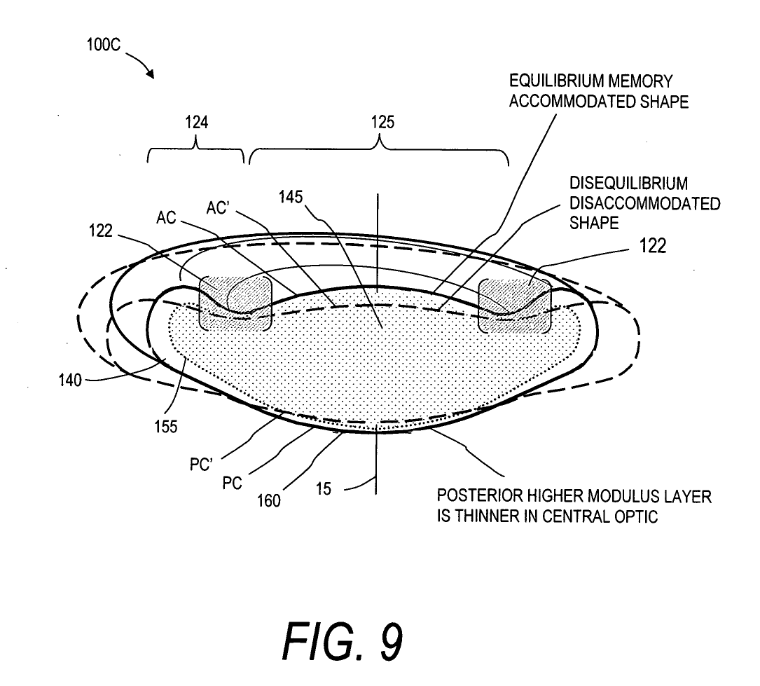

Now turning to FIG. 9, a preferred embodiment of elastomeric implant 100C is depicted, wherein the implant comprises an elastomer monolith that defines two elastomer block or body portions. The peripheral block portion indicated at 140 is substantially similar to implant body 100A of FIGS. 4A4B. The central optic portion 145 comprises an optic portion or lens for refracting light. The implant thus again defines an annular non-optic peripheral portion 124 around a central optic portion 125. The lens portion or block 145 is of an ultralow modulus polymer and can thus be actuated or adapted by the shape change of the annular body 140 and the transduction of forces across interface 155 between blocks or portions 140 and 145. In other words, the monolith 100C defines at least one interior, on-axis, rotationally symmetric interface 155 between at least two elastomeric block portions, 140 and 145, wherein each block portion has a different Young's modulus.

In finite element modeling, it has been found that the peripheral block portion 140 of a higher modulus can provide support of the lens capsule periphery wherein radial tensioning can cause substantial deformation of the anterior and posterior surfaces of the lens from AC to AC′ and PC to PC′. The elastomeric monolith has an equilibrium memory shape as in FIG. 9 that is deformable to the disequilibrium temporary shape (phantom view), wherein the memory shape provides a selected focusing power and the disequilibrium temporary shape provides a lesser focusing power. As described above, the elastomeric monolith moves from its equilibrium memory shape to the disequilibrium temporary shape of FIG. 9 in response to equatorial tensioning forces. In this embodiment, the central optic block portion 145 has a modulus ranging between about 1 KPa and 200 KPa. The peripheral block portion 140 has a modulus that is greater than the central block portion, wherein the modulus of the periphery can range from about 50 KPa to 400 KPa. In FIG. 9, the higher modulus material also can optionally extend in a thin form 160 across the posterior of the implant in which case it has an index of refraction that matches the central block portion 145.

Still referring to FIG. 9, finite element modeling has indicated that the annular arcuate region 122 at the periphery of the recess 120 (see FIG. 6B) advantageously amplifies the dioptric change of the lens under zonular tensioning—when compared to an elastomeric monolithic lens having a non-recessed shape (cf. FIG. 6A). The dioptric change may be as much as 2 to 4 diopters greater in an implant body with the arcuate surface region 122, together with a hyper-steepened anterior lens curvature as shown in FIGS. 7 and 9. The radially outward forces applied to the lens flatten the anterior and posterior curvatures across the entire lens surfaces. At the same time, the higher modulus peripheral block portion 140 can provide support of the lens capsule periphery and transduce forces to the ultralow modulus central lens portion. Alternatively, the higher modulus peripheral portion can comprise a plurality of symmetrical spaced apart regions about the periphery of the implant body.

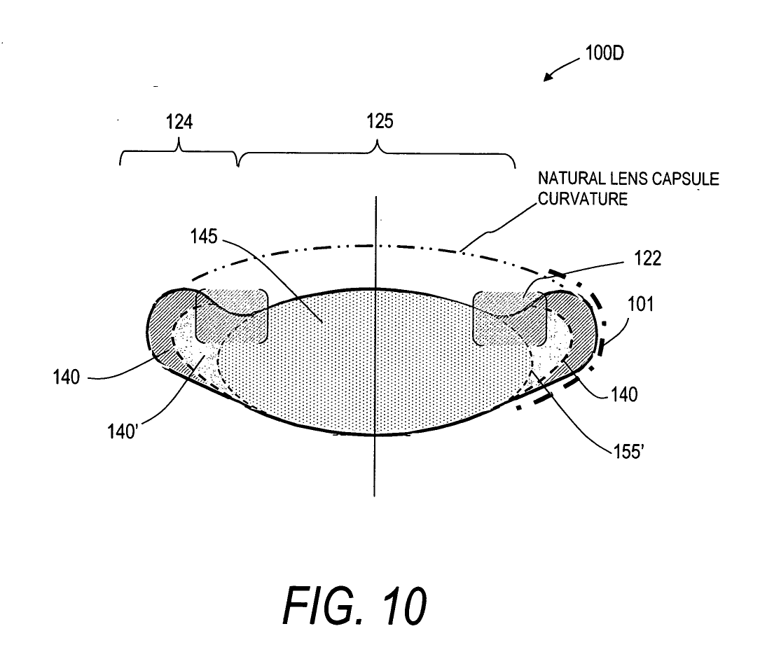

FIG. 10 depicts a similar embodiment of elastomeric implant 100D with annular non-optic peripheral portion 124 around central optic portion 125. The lens differs in that a gradient modulus is provided by a plurality of coupled, bonded together, elastomer body portions. The peripheral block portions indicated at 140 and 140′ are configured with a lower modulus in the inward direction and are coupled to an ultralow modulus central lens 145. Again, the on-axis, rotationally symmetric interfaces 155 and 155′ between the block portions are adapted for force transduction from the higher modulus block peripheral blocks to the lower modulus interior blocks. In any of the above embodiments, at least the peripheral block preferably is of a shape memory polymer as described previously.

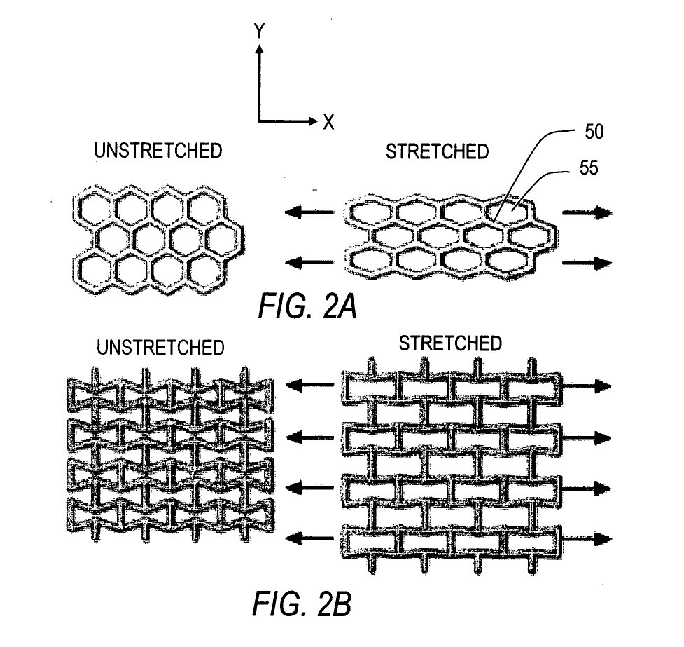

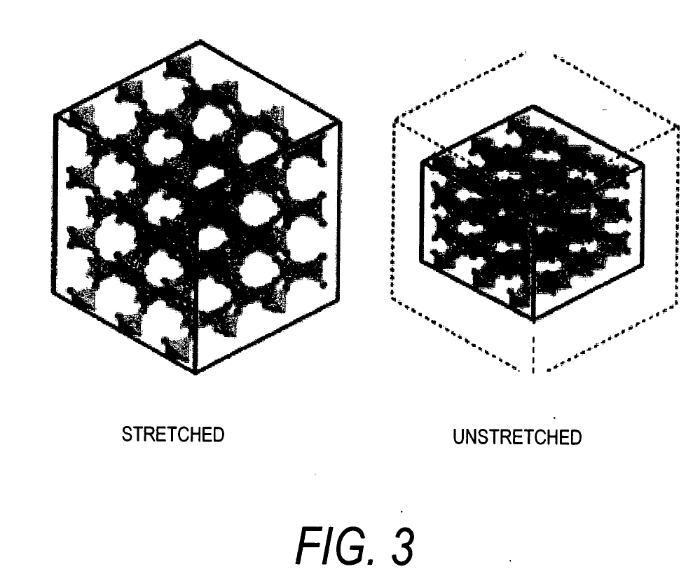

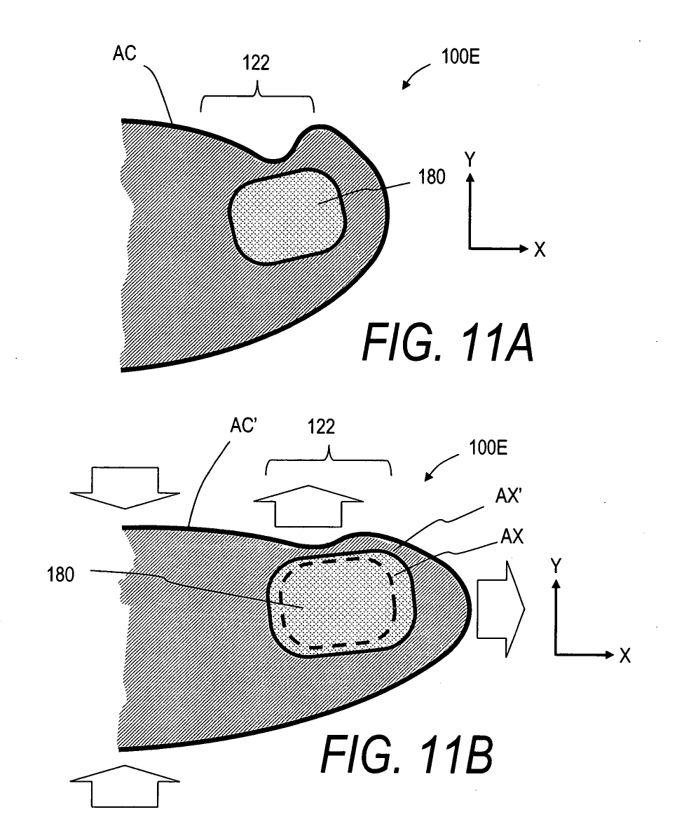

Another embodiment of implant 100E is shown in a schematic partial sectional view in FIGS. 11A and 11B, wherein the elastomeric monolith again has an anterior lens surface with a recessed region. This embodiment is configured to apply a different set of deforming forces to the anterior lens surface. Above, it was explained that the arcuate peripheral region 122 of the recessed portion can enhance anterior curvature deformation and accommodative amplitude in response to radial outward tensioning forces alone. The elastomeric monolith 100E of FIG. 11A is configured to further enhance the deformation and flattening of the anterior lens surface in the arcuate region 122 by altering the elastomer monolith's reaction forces in a symmetrical manner about the periphery of the recessed region. Of particular interest, the implant is configured with an auxetic material component indicated at 180 that can comprise a microfabricated open cell structure similar to that of FIG. 2B. The auxetic material can also be based on auxetic molecular level polymers as depicted in FIG. 3. Certain re-entrant auxetic foams have been developed and are known in the art and can be used for this purpose. Alternatively, the auxetic material, in either a micro- or nanoscale domain, can comprise a 360° annular element in the implant or its can consist of a plurality of radially symmetric elements. Microfabricated auxetic structures can be made by soft lithography means described below. As can be seen in FIG. 11B, the auxetic material moves from shape X to X′ upon radial outward forces along the x-axis thus causing y-axis forces to elevate the annular surface proximate the arcuate region 122 to thereby cause further flattening and alter the anterior lens curvature from AC to AC′ (see FIGS. 11A and 11B).

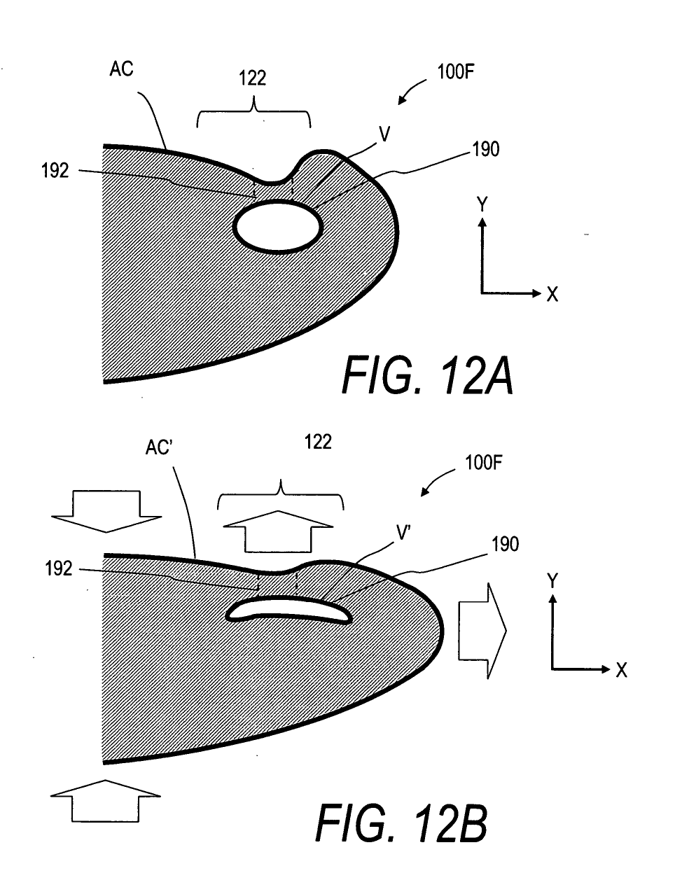

FIGS. 12A and 12B illustrate another embodiment of implant 100F that is based on similar principles as described in the previous embodiment. This embodiment again has a recessed anterior lens surface with an arcuate notch region indicated at 122. It has been found in finite element modeling that “inclusions” of at least one void or open cell 190 can enhance accommodative amplitude under radial outward tensioning forces—with an effect similar to that of auxetic materials. In FIG. 12A, the inclusion(s) can comprise a single annular void 190, a plurality of radially symmetric voids, or open cells of any suitable dimension. For example the open cells can be microfabricated by soft lithographic means. As can be seen in FIGS. 12A and 12B, the void 190 is altered in shape from shape V to V′ upon radial outward forces along the x-axis thus causing y-axis forces to elevate the anterior lens surface and cause a further flattening to anterior curvature AC′. A void region 190 in the non-optic portion can carry a gas, fluid or preferably communicate with the anterior chamber through a plurality of ports indicated at 192 in FIG. 12B.

In the exemplary embodiments of FIGS. 11A-12B, the implants can utilize a microfabricated auxetic polymer molecular structure or an open-cell microfabricated structure to provide anisotropic properties and modulus. The ordered structure of any embodiment of capsular shaping device can be microfabricated using soft lithography techniques. The structures can be microfabricated of a resilient polymer (e.g., silicone) by several different techniques, such as REM, μTM, MIMIC, SAMIM and several others—ollectively given the name of soft lithography. For example, microtransfer molding is used wherein an elastomeric polydimethylsiloxane (PDMS) stamp has patterned relief on its surface to generate features in the polymer. The PDMS stamp is filled with a prepolymer or ceramic precursor and placed on a substrate. The material is cured and the stamp is removed. The technique generates features as small as 250 nm and is able to generate multilayer systems that can be used to fabricate the implant of the invention. Replica molding is a similar process wherein a PDMS stamp is cast against a conventionally patterned master. A polyurethane or other polymer is then molded against the secondary PDMS master. In this way, multiple copies can be made without damaging the original master. The technique can replicate features as small as 30 nm. Another process is known as micromolding in capillaries (MIMIC) wherein continuous channels are formed when a PDMS stamp is brought into conformal contact with a solid substrate. Then, capillary action fills the channels with a polymer precursor. The polymer is cured and the stamp is removed. MIMIC can generate features down to 1 μm in size. Solvent-assisted microcontact molding (SAMIM) is also known wherein a small amount of solvent is spread on a patterned PDMS stamp and the stamp is placed on a polymer, such as photoresist. The solvent swells the polymer and causes it to expand to fill the surface relief of the stamp. Features as small as 60 nm have been produced. Various microfabricated polymeric “open” volume structures can be understood to be feasible from review of any text on soft lithography, for example as in Xia and Whitesides, Annu. Rev. Mater. Sci. 1998 28:153-84. In particular, FIGS. 3(h), 7(a) to 7(f) and 8(a) to 8(f) illustrate polymeric microstructures.

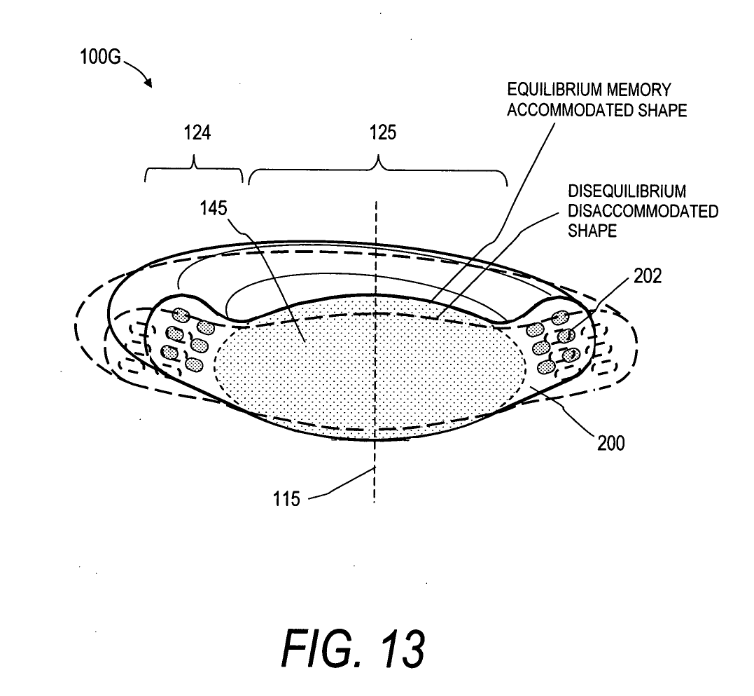

In another embodiment in FIG. 13, the implant 100F again has a recessed anterior lens surface with an arcuate notch region indicated at 122 and an ultralow modulus optic portion 145. This embodiment is similar to the embodiment of FIG. 12A with inclusions in the peripheral body portion 200. In this embodiment, negative stiffness inclusions 202 are provided with in the peripheral elastomer block 200 and are adapted amplify the limited zonular tensioning forces applied to the lens capsule. Negative stiffness materials can be of any of the types described in the references in the Summary of the Invention section above. The negative stiffness inclusions 202 are illustrated as a plurality of microscale elements but can be in any micro- or nanoscale (molecular) domain. It is believed by reducing the forces needed to radially deform the peripheral body portion, the accommodative amplitude can be enhanced under zonular tensioning. FIG. 13 shows the adaptive optic monolith in its equilibrium resting memory (accommodated) shape, with a phantom view of the monolith in a disequilibrium (disaccommodated) shape.

In the embodiment of FIG. 13, the monolithic elastomer portion 200 can have negative stiffness inclusions 202 are illustrated as a plurality of a shape memory material, such as a shape memory alloy or shape memory polymer that stores energy in a stressed shape that is fabricated into the block portion 200. In one example, a plurality of SMP elements 202 (FIG. 13) are provided in a strained configuration that stores energy (temporary shape). Following fabrication of the elastomer block 200, a thermal stimulus or other stimulus can be used alter the elements toward a memory unstrained configuration which will be resisted by the modulus of the elastomer block. In use, the restorative forces of the SMP elements can cooperate with and enhance the deformation of the elastomer block 200 under zonular tensioning. Such SMP elements can be adapted to apply restorative forces in a particular radial orientation.

Those skilled in the art will appreciate that the exemplary systems, combinations and descriptions are merely illustrative of the invention as a whole, and that variations in the compositions and designs fall within the spirit and scope of the invention. Specific characteristics and features of the invention and its method are described in relation to some figures and not in others, and this is for convenience only. While the principles of the invention have been made clear in the exemplary descriptions and combinations, it will be obvious to those skilled in the art that modifications may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

1. An intraocular lens comprising a monolithic elastomer body configured for 360° elastic intracapsular engagement with a lens capsule periphery wherein the elastomer body has an anisotropic elastic modulus.

2. An intraocular lens as in claim 1 wherein the elastomer body has an optical axis, the body including a central optic portion having a first elastic modulus and at least one on-axis symmetric peripheral body portion having a different elastic modulus.

3. An intraocular lens as in claim 2 wherein the elastomer body defines an on-axis radially symmetric gradient in elastic modulus.

4. An intraocular lens as in claim 1 wherein at least a portion of the lens is of a shape memory polymer.

5. An intraocular lens as in claim 2 wherein a substantial region of the central optic portion defines an elastic modulus of less that 400 KPa.

6. An intraocular lens as in claim 2 wherein a substantial region of the central optic portion defines an elastic modulus of less that 200 KPa.

7. An intraocular lens as in claim 2 wherein a substantial region of the central optic portion defines an elastic modulus of less that 100 KPa.

8. An intraocular lens as in claim 2 wherein the central optic portion has a recessed anterior lens surface in relation to a peripheral body portion.

9. An intraocular lens as in claim 1 wherein a peripheral body portion includes at least one of auxetic materials, inclusions of open cells and inclusions of negative stiffness materials.

10. An intraocular lens as in claim 2 wherein the central optic portion includes first and second spaced apart lenses.

11. An intraocular lens as in claim 10 wherein at least one lens is modular and de-matable.

12. An intraocular lens comprising at least in part an auxetic material.

13. An intraocular lens as in claim 12 wherein the auxetic material is a foam.

14. An intraocular lens as in claim 12 wherein the auxetic material is defined by a microscale microfabrication domain.

15. An intraocular lens as in claim 12 wherein the auxetic material is a soft lithography microfabrication.

16. An intraocular lens as in claim 12 wherein the auxetic material is defined by a nanoscale molecular domain.

17. An intraocular lens as in claim 12 wherein the auxetic material has a node and fibril structure.

18. An intraocular lens as in claim 12 wherein the auxetic material has a radially symmetric orientation.

19. An intraocular lens as in claim 12 wherein the auxetic material is configured to respond to radial outward forces by expansion in a transverse direction.

20. An intraocular lens as in claim 12 wherein the auxetic material is within an annular body region spaced outwardly from an optical axis of the lens.

21. An intraocular lens as in claim 20 wherein the auxetic material is within radially symmetric spaced apart portions of the annular body region.

22. An intraocular lens defining a central optic portion and a peripheral non-optic portion configured for 360° intracapsular engagement with a lens capsule periphery wherein a deformable anterior lens surface is recessed therein.

23. An intraocular lens as in claim 22 wherein 100% of the surfaces of the peripheral non-optic portion that intracapsularly engage the lens capsule are omni-directionally elastic.

24. An intraocular lens as in claim 22 wherein the central optic portion is at least in part a shape memory polymer.

25. An intraocular lens as in claim 22 wherein the peripheral non-optic portion is at least in part a shape memory polymer.

27. An intraocular lens as in claim 22 wherein the peripheral non-optic portion is at least in part of a material selected from the class consisting of auxetic materials, materials having inclusions of open cells and materials having negative stiffness inclusions.

28. An intraocular lens as in claim 27 wherein the selected material is within radially symmetric regions of the lens.

29. An intraocular lens as in claim 22 wherein the central optic portion is modular and de-matable from the peripheral non-optic portion.

30. A method of enabling lens accommodation, comprising:
providing an annular body configured for 360° elastic intracapsular engagement of a lens capsule periphery, the annular body including anisotropic materials comprising at least one of auxetic materials, material with inclusions of open cells or materials with inclusions of negative stiffness; and
coupling an elastomeric lens centrally to said annular body, wherein radial outward forces on the annular body cause transduction of radial first deforming forces to a surface curvature of the elastomeric lens, and wherein said radial outward forces cause the anisotropic materials to apply radially-transverse second deforming forces to said lens curvature.

31. A method of enabling intraocular lens accommodation as in claim 30 wherein the first deforming forces flatten at least one of anterior or posterior surface curvatures.

32. A method of enabling intraocular lens accommodation as in claim 30 wherein the second deforming forces flatten a periphery of at least one of anterior or posterior surface curvatures.

33. A method of fabricating an intraocular accommodative device, comprising providing an annular body configured for 360° elastic intracapsular engagement of a lens capsule periphery, the annular body including anisotropic materials comprising at least one of auxetic materials, materials having inclusions of open cells or materials having negative stiffness inclusions.

34. A method of fabricating an intraocular accommodative device as in claim 33 further comprising coupling an elastomeric lens centrally to said annular body, the annular body configured for applying both radial outward deforming forces and radially-transverse deforming forces to the lens.

35. A method of fabricating an intraocular accommodative device as in claim 33 wherein the annular body is provided in a shape memory polymer.

36. A method of fabricating an intraocular accommodative device as in claim 33 wherein the elastomeric lens is provided in a shape memory polymer.
